(19) United States
(12) Patent Application Publication
(10) Pub. No.: US 2021/0060264 A1
(43) Pub. Date: Mar. 4, 2021

(54) INJECTION NEEDLE HAVING VARYING CALIBER

(71) Applicant: David Vincent Martini, DOUGLAS, GA (US)

(72) Inventor: David Vincent Martini, DOUGLAS, GA (US)

(21) Appl. No.: 16/558,024

(22) Filed: Aug. 30, 2019

(51) Int. Cl. A61M 5/32; A61M 5/31

(52) U.S. Cl. CPC A61M 5/3286; A61M 5/3269; A61M 2210/0681; A61M 5/3148; A61M 5/329

(57) ABSTRACT

An injection needle including a guide portion configured to have a first outer diameter, and a delivery portion, configured with a tip to penetrate tissue and deliver a medication, and to have a second outer diameter smaller than the first outer diameter, extending from a distal end of the guide portion, wherein the guide portion is configured to have a bend proximate the distal end such that the delivery portion extends at a predetermined angle from a line of the guide portion opposite the bend. An injection needle including means to scavenge medication and fluids from the patient and procedure site using a suction port, conduct along needle body via conduit, and evacuate to outside of patient suction unit via exit port. A tube may be connected to exit port and connect to external collection unit such as a medical suction device.

### CROSS-REFERENCE TO RELATED APPLICATIONS

This application claims priority from U.S. Provisional Application No. 62/119,530, filed on Feb. 23, 2015.

### FIELD OF INVENTION

The present general inventive concept relates generally to injection needles, and, more particularly, to an injection needle having [1] a varying diameter such that a wider needle body provides added structural support for a narrower needle tip and [2] structures to scavenge blood, body secretions, and spilled injection medication.

### BACKGROUND

An injection needle is typically used by a health care professional (e.g., a surgeon or nurse) to administer pharmaceutical drugs to a patient. Often, circumstances require the injection needle to be administered where physical spacing is limited. Large diameter (smaller needle gauge number) injection needles allow for precision administration of the pharmaceuticals due to their low flexibility; however, the larger the injection needle, generally the more pain and tissue damage the patient can incur. Small diameter (large needle gauge number) injection needles offer high flexibility allowing for improved maneuverability and lower tissue damage/pain. Yet, because small diameter injection needles are flexible, it is more difficult to administer the pharmaceuticals to the precise location at which the health care professional intends. Additionally, during the injection of medication, excess medication will spill out from the tissues and have an adverse effect on the patient. Blood and body fluids may also need to be evacuated, for example to permit safe visualization. An additional inventive concept includes in proximity to the needle injection tip a scavenger capacity via a suction port, a conduit to transfer the aspirate, and an exit port to attach to an external suction. For example, FIG. 1 illustrates a conventional injection needle 100 having a small diameter and a high flexibility. The thin needle 104 extends from a hub 102 that may be used to couple the injection needle 100 to a syringe or other such medication injection device. In an example procedure such as delivering a medication to the sinuses of a patient, such a small diameter and flexible injection needle may be needed for the tissue of the sinuses that will have the medication injected. However, because the sinuses may need to be accessed through the patient's nostrils, the flexibility of such a needle presents problems as it may lack a stiffness that may be needed to guide the needle through the nostrils to the target region of the sinuses. Thus, a small diameter needle with a more rigid body to guide the needle may be desired. Additionally, spilled medication and body fluids may be safely evacuated.

### BRIEF SUMMARY

The present general inventive concept provides an injection needle including a first needle portion having a first outer diameter, and a second needle portion having a second outer diameter smaller than the first outer diameter and a tip configured to penetrate tissue of a patient to deliver medication. The present general inventive concept provides an apparatus to scavenge, conduct material safely away from injection site, and deposit to an external suction device.

Additional aspects and advantages of the present general inventive concept will be set forth in part in the description which follows, and, in part, will be obvious from the description, or may be learned by practice of the present general inventive concept.

The foregoing and/or other aspects and advantages of the present general inventive concept may be achieved by an injection needle including a first needle portion having a first outer diameter, and a second needle portion having a second outer diameter, the second outer diameter being smaller than the first outer diameter, wherein the second needle portion has a tip configured to penetrate a soft tissue. The foregoing and/or other aspects and advantages of the present general inventive concept may be achieved by an injection needle including an apparatus to scavenge, conduct material safely away from injection site, and deposit to an external suction device.

The foregoing and/or other aspects and advantages of the present general inventive concept may also be achieved by an injection needle including a guide portion configured to have a first outer diameter, and a delivery portion, configured with a tip to penetrate tissue and deliver a medication, and to have a second outer diameter smaller than the first outer diameter, extending from a distal end of the guide portion, wherein the guide portion is configured to have a bend proximate the distal end such that the delivery portion extends at a predetermined angle from a line of the guide portion opposite the bend.

The foregoing and/or other aspects and advantages of the present general inventive concept may also be achieved by an injection needle including a hub portion configured to mate with a corresponding hub portion of a medication delivery device, and a needle attached at a proximal end to the hub and configured to have a first outer diameter extending in a longitudinal direction from the hub, the first outer diameter having a bend portion proximate a distal end of the needle, the bend portion having a predetermined angle matching a corresponding tissue path through which the needle will pass to enter a targeted region of the patient, the needle including a distal end configured to have a second outer diameter smaller than the first outer diameter, the second diameter terminating to a needle tip for delivery of medication into the targeted region, the first diameter being specifically sized to push away bodily tissue in the tissue path a distance sufficient to provide an opening for the second diameter to enter the targeted region at the predetermined angle to deliver medication to the targeted region.

The injection needle may further include a ridged portion formed proximate a point at which the tapering portion begins from the first outer diameter, the ridged portion having a third outer diameter larger than the first outer diameter specifically sized to abut bodily tissue at an end of the tissue path to inhibit the needle tip from penetrating beyond the targeted region.

Additional features and aspects will be apparent from the following detailed description, the drawings, and the claims.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 illustrates a conventional injection needle having a small diameter and a high flexibility;

FIG. 2 illustrates an injection needle according to an example embodiment of the present general inventive concept;

FIG. 3 illustrates an injection needle according to another example embodiment of the present general inventive concept;

FIG. 4 illustrates an injection needle according to yet another example embodiment of the present general inventive concept;

FIG. 5 illustrates a cross section of the injection needle illustrated in FIG. 4;

FIG. 6 illustrates a cross section of an injection needle according to another example embodiment of the present general inventive concept; and

FIGS. 7-9 illustrate the fabrication of an example injection needle from a single needle body according to an example embodiment of the present general inventive concept.

FIG. 10 illustrates the components and concepts of the fluid suction and evacuation inventive concept. Port #901, (suction port) to scavenge spilled medication and body fluids, a conduit to the exit port #902 (exit port), which is attached to an external suction device for removal.

### DETAILED DESCRIPTION

Reference will now be made to various example embodiments of the present general inventive concept, examples of which are illustrated in the accompanying drawings and illustrations. The example embodiments are described herein in order to explain the present general inventive concept by referring to the figures.

The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. The described progression of processing operations described are merely examples, however, and the sequence of operations is not limited to that set forth herein and may be changed as is known in the art, with the exception of operations necessarily occurring in a certain order. Also, description of well-known functions and constructions may be omitted for increased clarity and conciseness.

Note that spatially relative terms, such as “up,” “down,” “right,” “left,” “beneath,” “below,” “lower,” “above,” “upper” and the like, may be used herein for ease of description to describe one element or feature's relationship to another element(s) or feature(s) as illustrated in the figures. Spatially relative terms are intended to encompass different orientations of the device in use or operation in addition to the orientation illustrated in the figures. For example, if the device in the figures is turned over or rotated, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

Various example embodiments of the present general inventive concept, as described herein, provide an injection needle of varying caliber such that a first portion of the injection needle is more rigid than a second portion of the injection needle having a tip that is used to inject medication into a patient. An example embodiment may provide an injection needle including a first needle portion having a first outer diameter, and a second needle portion having a second outer diameter smaller than the first outer diameter and a tip configured to penetrate tissue of a patient to deliver medication. Thus, a user administering the medication is able to take advantage of the higher rigidity of the larger diameter portion to guide the smaller diameter portion to the desired injection point to deliver the medication. To provide even further advantage to the user administering the medication in situations that present difficult to reach places such as sinuses that are accessed through a patient's nostrils, an example embodiment of the present general inventive concept may provide injection needle including a guide portion configured to have a first outer diameter, and a delivery portion, configured with a tip to penetrate tissue and deliver a medication, and to have a second outer diameter smaller than the first outer diameter, extending from a distal end of the guide portion, wherein the guide portion is configured to have a bend proximate the distal end such that the delivery portion extends at a predetermined angle from a line of the guide portion opposite the bend.

Various example embodiments herein may describe a first portion of an injection needle that has a first outer diameter greater than a second outer diameter of a second portion of the injection needle. The first portion having the larger outer diameter may be interchangeably referred to as a “guide” portion, as the increased rigidity of the larger diameter portion may be used to more effectively guide the injection needle through various cavities, openings, etc., of the body of the patient than the conventional needle having only one continuous outer diameter, which may be too flexible for such guidance due to the required injection tip size. Similarly, the second portion having the smaller diameter and injection tip may be interchangeably referred to as a “delivery” portion, as the increased flexibility of the smaller diameter portion may be more effective in injecting the tip into the actual desired tissue.

For example, the injection needle can be specifically configured to have a first outer diameter extending in a longitudinal direction from a hub portion, the first outer diameter having a bend portion proximate a distal end of the needle to provide a predetermined angle matching a corresponding tissue path of the patient through which the needle will pass. The first diameter is sized to push bodily tissue in the tissue path away from the tissue path a distance sufficient to create an opening for the second diameter to enter the targeted region at the predetermined angle to deliver medication to the targeted region without causing the second diameter to bend.

As described herein, the present general inventive concept relates to an injection needle of varying caliber (i.e., outer diameter/gauge). Specifically, the present general inventive concept relates to an injection needle that includes a smaller diameter needle proximate a needle tip, and a larger diameter needle as the main shaft. Varying diameter injection needles have a variety of advantages, such as increased precision and a decrease in tissue damage, which correspond with low pain upon injection.

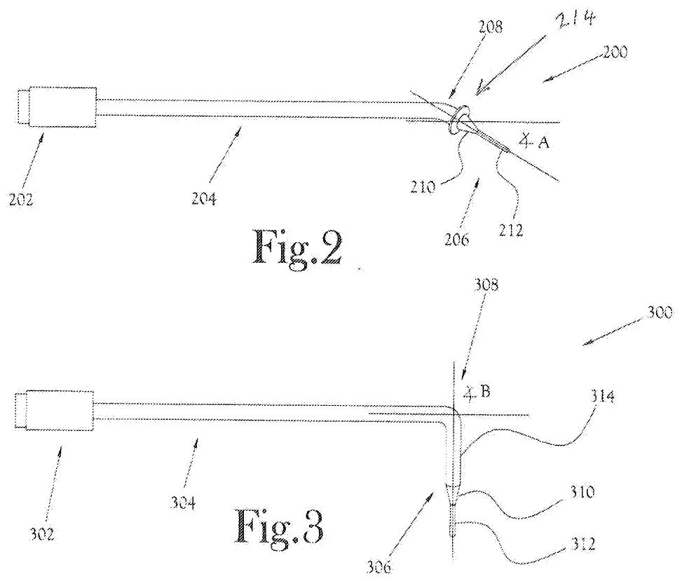

FIG. 2 illustrates an injection needle according to an example embodiment of the present general inventive concept. Referring now to FIG. 2, an example injection needle 200 is illustrated. In this example embodiment, the injection needle 200 includes a hub 202, a main shaft 204, and a needle end 206. The illustrated example embodiment is configured for use as a nose/sinus local anesthesia injection needle. The illustrated example embodiment depicts an injection needle 200 configured to be attached to a syringe or other like medical device; however, it is contemplated as part of the present general inventive concept that a syringe or like medical device can be an integral part of the injection needle 200. In some example embodiments, the injection needle 200 may be configured to be reusable. In other example embodiments, the injection needle 200 may be configured to be disposable after a single use.

In the example embodiment illustrated in FIG. 2, the hub 202 is a hub configured to mate with a corresponding similar hub of, for example, a syringe or other medication delivery device. In some example embodiments, the hub 202 may be configured to mate with a syringe. In other example embodiments, the hub 202 may include a Luer taper connection. In some example embodiments, the hub 202 may be a Luer-Lok™ hub configured to mate with other similar Luer-Lok™ devices. In the illustrated example embodiment, the hub 202 is secured to the main shaft 204.

In the example embodiment illustrated in FIG. 2, the main shaft 204 has an outer diameter to allow sufficient precision by the user when positioned at the site of injection on a patient. In some example embodiments, the main shaft may be a 16-gauge needle. Also, in some example embodiments, the length of the main shaft 204 can vary depending on the application. In some example embodiments, the length of the main shaft 204 may be about 80 millimeters. At the opposite end of the main shaft 204 from the hub 202 is the needle end 206. As illustrated in FIG. 2, in this example embodiment, not only is the needle end 206 of a significantly smaller outer diameter than that of the main shaft 204, the needle end 206 is also angled away from most of the main shaft 204. This angle is facilitated by a bend 208 provided proximate the distal end of the main shaft 204, where the needle end 206 is located. It is noted that in this example embodiment the outer diameter of the main shaft 204 is maintained on both sides of the bend 208.

The needle end 206 is configured to transition the injection needle 200, specifically the main shaft 204, to a smaller diameter needle (larger gauge number, relative to the main shaft 204 gauge) so that the needle end 206 has more flexibility than the main shaft 204 (small gauge number, relative to the needle end 206), as well as a smaller presence in the tissue into which it will be injected. In the illustrated example embodiment, the needle end 206 a tapered section 210 leading to a tip 212. In various example embodiments, the tapering of the tapered section 210 may begin close to the bend 208, or may begin a predetermined distance past the bend 208. A safety stop may be provided proximate to the bend 208, near which the tapering from the main shaft 204 may begin.

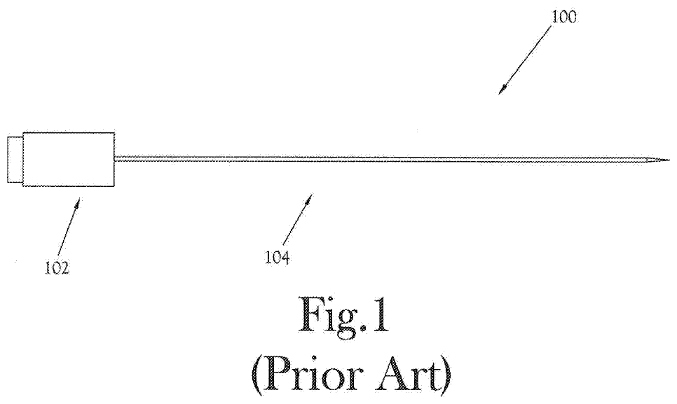

As previously discussed, in some example embodiments, the needle end 206 can make an angle A with the main shaft 204 beginning at the bend 208. Depending on certain applications, the angle A can range from about 0 degrees to about 90 degrees. In the example embodiment illustrated in FIG. 1, which includes sinus operations as a possible use, the angle A is about 37 degrees.

The safety stop 214 may be a lip, ridge, etc., on the injection needle 200 that prevents the user of the injection needle 200 from inserting the injection needle 200 too far into the tissue of a patient. The safety stop 214 may be sized to physically stop the penetration of the injection needle 200 past the safety stop 214. The safety stop 214 may be configured to allow the user of the injection needle 200 to inject the injection needle 200 at the proper depth in the patients tissue even when the user cannot physically see the injection site. In the illustrated example nose/sinus local anesthesia injection needle embodiment, the safety stop 214 has an outer diameter of about 1.5 millimeters. In various example embodiments, the safety stop 214 may be adhered to the main shaft 204 by a variety of methods and/or adhesives, and may be formed of a different material than the injection needle 200. Various other example embodiments may omit the safety stop 214 entirely, and may rely on the tapered section 210 or the diameter of the main shaft 204 to provide a similar stopping effect.

In some example embodiments, the injection needle 200 may transition directly to the tip 212, which has a smaller diameter than the main shaft 204, immediately after the safety stop 214. In the example embodiment illustrated in FIG. 2, the needle end 206 includes a tapered section 210 between the safety stop 214 and the tip 212. At the end closest to the main shaft 104, the tapered section 210 has an outer diameter substantially similar to the outer diameter of the main shaft 204. The tapered section 210 decreases in diameter (i.e. numerically increases in needle gauge) along its length in a direction toward the tip 212. In some example embodiments, the tapered section 210 improves the strength of the injection needle, making the needle end 206 more resilient to inadvertent breaking. In the illustrated nose/sinus local anesthesia injection needle embodiment, the tapered section 210 is about 5 millimeters in length. Also, in the illustrated nose/sinus local anesthesia injection needle embodiment, the largest gauge portion of the tapered section is about a 16-gauge needle, and the smallest gauge portion of the tapered section 210 is about a 27-gauge needle.

The tip 212 has a smaller outer diameter than that the main shaft 204. In some example embodiments, the tip 212 is an integral part of the injection needle 200. In other example embodiments, the tip 212 can be a modular portion of the injection needle 212. In the illustrated nose/sinus local anesthesia injection needle embodiment, the needle tip 212 can be about 10 millimeters in length and about a 27-gauge needle.

FIG. 3 illustrates an injection needle according to another example embodiment of the present general inventive concept. The example embodiment injection needle 300 illustrated in FIG. 3 is configured for use as a tongue local anesthesia injection needle. Similar to the injection needle 200 illustrated in FIG. 2, in some example embodiments the injection needle 300 may be configured to be reusable. In other example embodiments, the injection needle 300 may be configured to be disposable after a single use. The injection needle 300 shares some common features with the injection needle 200 illustrated in FIG. 2. Accordingly, the injection needle 300 includes a hub 302, a main shaft 304, and a needle end 306. The hub 302 and most of the main shaft 304 are substantially similar to the hub 202 and the main shaft 204 of the injection needle 200 illustrated in FIG. 2.

In the example embodiment illustrated in FIG. 3, the needle end 306 includes a tapered portion 310, a needle tip 312, and a curved portion, or bend, 308 of the main shaft 304. Although no safety stop is illustrated in the example embodiment illustrated in FIG. 3, various example embodiments may also include such a feature, which may serve a similar purpose to the safety stop 214 of the example embodiments illustrated in FIG. 2. The needle tip 306 is oriented in a manner to facilitate precise injection into a tongue of a patient. Accordingly, the needle end 306 forms an angle B of about 90 degrees with the main shaft 304.

The tapered portion 310 is also substantially similar to the tapered portion 210 of FIG. 2. The tapered portion 310 begins at an outer diameter of the main shaft 304 and tapers to a diameter substantially similar to that of the tip 312. In the illustrated embodiment, the tapered portion 310 has a length of about 3.5 millimeters, but other various example embodiments may include tapered portions of different sizes, or eliminate the tapered portion altogether.

The tip 312 has a smaller diameter than the main shaft 304. In various example embodiments, the tip 312 can be about 4 millimeters in length and have a gauge of about 27. Other various example embodiments may include different lengths and gauges.

The curved portion 308 of the main shaft is disposed proximate to the distal end of the main shaft 304, and an extension 314 of the main shaft 308 extends at the angle B from the curved portion 308. The extension 314 of the main shaft 304 beyond the curved portion 308 of the main shaft 304 has a substantially similar outer diameter as that of the main shaft 304. In various example embodiments, the extension 314 of the main shaft 304 beyond the curved portion 308 of the main shaft 304 helps give the injection needle 300 the rigidity it needs for proper injection. In some embodiments, the extension 314 of the main shaft 304 beyond the curved portion 308 is about 7.5 millimeters in length.

Although the example embodiments in FIGS. 2-3 illustrate an injection needle device, a host of other configurations are possible in other various example embodiments of the present general inventive concept. For example, a large variety of lengths and diameters of the tapered diameter injection needles 200, 300 can be created depending on the medical application. Further, the angles the needle ends 206, 306 make with the main shafts 204, 304 can be modified for many different specific applications.

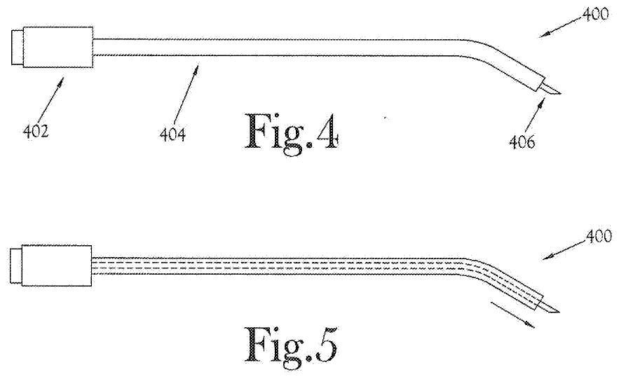

FIG. 4 illustrates an injection needle according to yet another example embodiment of the present general inventive concept. The example embodiment of the injection needle 400 illustrated in FIG. 4 is similar in many aspects to the injection needles 200 and 300 illustrated in FIGS. 2-3, having a hub 402 from which a main shaft 404, or guide portion, extends and has a first diameter. However, the needle end 406, or delivery portion, extending from a distal end of the main shaft 404, and having a second diameter smaller than the first diameter, extends throughout the length of the main shaft 404. FIG. 5 illustrates a cross section of the injection needle illustrated in FIG. 4. As illustrated in FIG. 5, the delivery portion extends all the way to the hub 402, and is enclosed inside the main shaft 404 in a sleeve type configuration. Such an arrangement may be more convenient to construct in some situations, with the smaller diameter and more flexible delivery portion simply slid inside the main shaft 404 having the larger diameter.

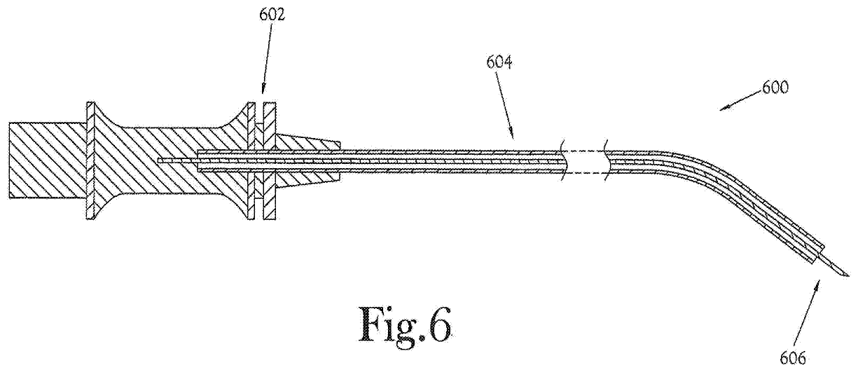

FIG. 6 illustrates a cross section of an injection needle according to another example embodiment of the present general inventive concept. The example embodiment illustrated in FIG. 6 is simply a slightly more detailed look of an injection needle similar to the embodiment illustrated in FIGS. 4-5. As illustrated in FIG. 6, a delivery portion 606 of the injection needle 600 extends completely through, and is enclosed by, the guiding portion 604. Both the guiding portion 604 and the delivery portion 606 are fixed to the hub 602. As previously described, in various example embodiments the entirety of the injection needle 600 may be disposable. In other various example embodiments, the delivery portion 606 may be disposable, while the hub 602 and guide portion 604 arrangement are reusable. In various example embodiments, the guide portion 604 and delivery portion 606 may be fixed to the hub 602 in a variety of different ways, such as, for example, a friction fit. The hub 602 may be assembled of two or more parts, to provide access for friction fitting, or other types of adherence, of the guide portion 604 and/or delivery portion 606 to the hub 602. Although the inner bore of the guide member 604 is exaggerated in FIG. 6 for clarity of structure, in various example embodiments the guide member 604 is configured such that the delivery member 606 has little to no room for lateral movement inside the guide member 604. In other example embodiments, the inner bore of the guide member 604 may be configured so as to accept a variety of delivery members 606 having different outer diameters such that different gauges of delivery portions 606 may be used for different procedures while using the same hub 602 and guide portion 604 arrangement. In such an embodiment, differently configured hubs or hub portion may be selectively provided to form a friction fit with the desired size of needle used as the delivery portion 606. Also, different example embodiments may provide different lengths of the delivery portion 606 that extend past the end of the guide portion 604, depending upon the procedure and/or tissue associated with the injection of medication.

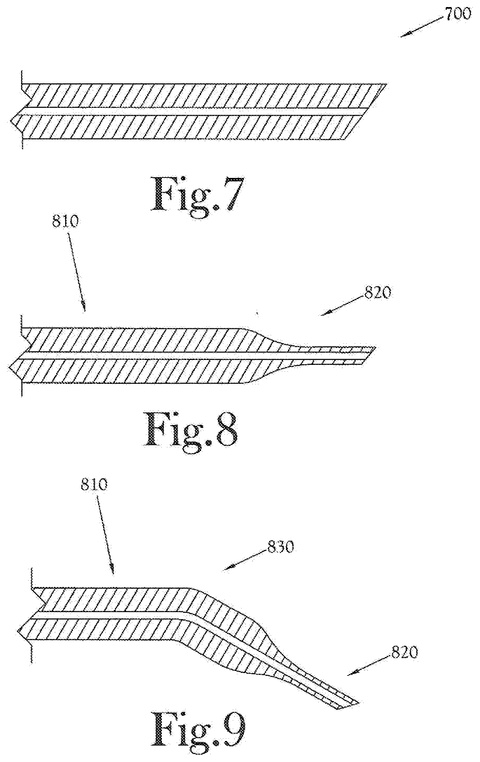

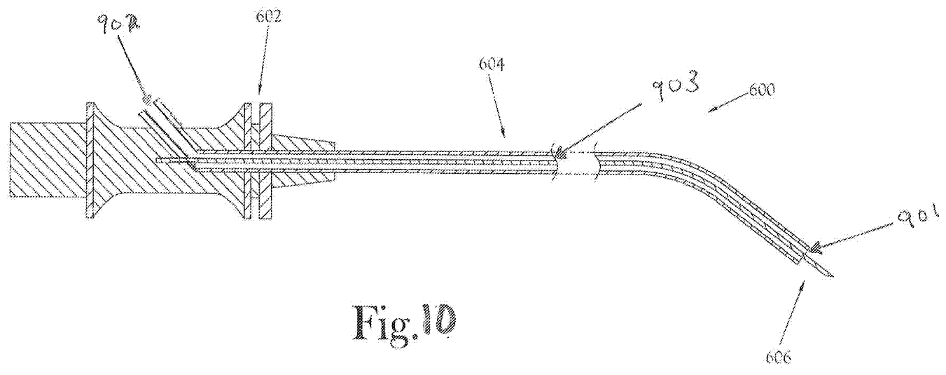

As previously described, injection needles according to the present general inventive concept may provide, according to various example embodiments, smaller diameter delivery portions that may be formed separately from the guide portions, such as in an enclosed or attached configuration, or may be formed integrally, or from the same body. FIGS. 7-9 illustrate the formation of an example injection needle from a single needle body according to an example embodiment of the present general inventive concept. It is understood that the process illustrated in FIGS. 7-9, and associated description herein, is merely one example of how an injection needle according to the present general inventive concept may be formed and/or configured. FIG. 7 illustrates a needle body 700 having a first outer diameter. A predetermined portion of a distal end of the needle body 700 may be milled down, or otherwise reduced to produce the desired outer diameter and length of the delivery portion. FIG. 8 illustrates the guide portion 810 of the injection needle, which has the first diameter, and the delivery portion 820 having the second outer diameter. As also illustrated in FIG. 8, a tapered portion may be provided at the transition from the guide portion 810 to the delivery portion 820 to provide added strength and prevention against the delivery portion 820 breaking off from the guide portion 810. FIG. 9 illustrates the injection needle after a bend 830 is performed so that the delivery portion 820 forms a desired angle with a longitudinal axis of the guide portion 810 on the other side of the bend 830. Alternatively, the bend 830 may be formed before the forming of the outer diameter of the delivery portion 820. The bend 830 may be formed such that the outer diameter of the guide portion 810 is maintained for a predetermined distance between the bend 830 and the delivery portion 820.

As discussed in relation to various example embodiments described herein, the bend or bend portion of the injection needle may be configured such that the relatively sturdier larger diameter guide portion is able to push away bodily tissue in a tissue path so as to provide access for the smaller diameter needle tip to enter a targeted body region at a predetermined angle to deliver medication to the targeted region. The present general inventive concept may include several such angles, such as the examples discussed herein, but is not limited to any such configuration. For example, in a medical procedure in which a medical practitioner or user wishes to deliver medication through the injection needle to, for example, the frontal sinus of a patient, the location of the sinus with respect to anatomical structures such as the middle turbinate, ethmoid bulla, middle uncinate, etc., results in a situation in which the medical practitioner needs to both reach around anatomic corners and displace anatomic structures. For example, the user may need to displace such structures as the middle turbinate and middle uncinate, and angle around (or “wrap” around), for example, the middle turbinate and ethmoid bulla, for accurate placement of the needle tip in or at the sinus. An injection needle according to various example embodiments of the present general inventive concept allows the user to both displace and reach around such structures, as the case may be. As another example procedure, a medical practitioner may need to access the maxillary sinus. Similar to the procedure employed to access the frontal sinus, to accurately access the maxillary sinus the configuration of the injection needle according to the present general inventive concept allows the user to displace the middle turbinate and angle around the middle uncinate, and the angle of the bend of the injection needle allows access to the maxillary sinus, which is laterally located to the nasal cavity. As another example, to more conveniently and accurately access the sphenoid recess (or sinus), which is located posteriorly in the nasal cavity, the length, rigidity, and angulation of the injection needle of the present general inventive concept allows the user to reach such a deep cavity by angling around the middle uncinated while displacing the middle turbinate and superior turbinate to expose the sphenoid sinus. As yet another example, to conveniently and accurately access the ethmoid sinus, the length, rigidity, and angulation of the injection needle of the present general inventive concept allows the user to reach and access the ethmoid sinuses while pushing the middle turbinate out of the way of the injection needle. As previously described herein, an angle of 37 degrees of the needle tip relative to the guide portion on the opposite end of the bend from the needle tip may be used to access one or more of these described sinuses. In various example embodiments, the injection needle may be configured with bends of different angles.

Also, as described herein, the injection needle of the present general inventive concept is no limited to applications related to nasal passages and sinuses, but may be used in a host of procedures in which such a rigid length of a guide portion aids in the placement of an angled delivery portion having a needle tip. For example, in a procedure in which a medical practitioner may need to deliver medication to a back area of the tongue of a patient, the configuration of the injection needle according to an example embodiment of the present general inventive concept provides a sufficient length to span the length of the tongue and deposit the medication, such as anesthesia at tongue base and middle and/or posterior aspects of the tongue, uvula, pharynx area, etc. In an example embodiment, a 90-degree angle at the bend of the injection needle may be desired to perform such a procedure.

While the various example embodiments illustrated and/or discussed herein have been discussed in terms of human patients, it is understood that various example embodiments of the present general inventive concept may be configured for use in veterinary procedures without departing from the scope and spirit of the present general inventive concept.

Various example embodiments of the present general inventive concept may provide an injection needle including a guide portion configured to have a first outer diameter, and a delivery portion, configured with a tip to penetrate tissue and deliver a medication, and to have a second outer diameter smaller than the first outer diameter, extending from a distal end of the guide portion, wherein the guide portion is configured to have a bend proximate the distal end such that the delivery portion extends at a predetermined angle from a line of the guide portion opposite the bend. The guide portion may be configured to have the first outer diameter at both sides extending from the bend. The injection needle may further include a hub provided at a proximal end of the guide portion; the hub being configured to mate with a corresponding hub to connect the injection needle to a medication delivery device. The guide portion and the delivery portion may be formed from a common body. The delivery portion may be configured to be tapered from the distal end of the guide portion to a predetermined point on the delivery portion. The injection needle may further include a ridged portion having a third outer diameter larger than the first outer diameter, and provided at the distal end of the guide portion, to serve as a safety stop for the delivery portion. The ridged portion may be a separate body adhered to the injection needle. The ridged portion may be formed of a separate material than the injection needle. The injection needle may further include a ridged portion proximate the distal end of the guide portion, the ridged portion having a third outer diameter larger than the first outer diameter, such that the ridged portion serves as a safety stop for the delivery portion. The injection guide portion may be more rigid than the delivery portion. The guide portion and the delivery portion may be separately formed. The delivery portion may extend through the entirety of the guide portion, the guide portion being configured as a sleeve surrounding the delivery portion. The injection needle may further include a ridged portion having a third outer diameter larger than the first outer diameter, and provided proximate the distal end of the guide portion, to serve as a safety stop for the delivery portion. The guide portion may be more rigid than the delivery portion. Both the guide portion and the delivery portion may be fixed to a hub at a proximal end of the guide portion. The delivery portion may be adhered to the guide portion.

Various example embodiments of the present general inventive concept may provide an injection needle including a first needle portion having a first outer diameter, and a second needle portion having a second outer diameter, the second outer diameter being smaller than the first outer diameter, wherein the second needle portion has a tip configured to penetrate a soft tissue. The injection needle may include a hub mounted to the first needle portion; the hub being configured to mate with other similar hubs. The second needle portion may form an angle with the first needle portion. In some example embodiments, the angle may be approximately 37 degrees. In some example embodiments, the angle may be approximately 90 degrees. The injection needle may further include a safety stop positioned at an opposite end of the second needle portion from the tip, the safety stop having a larger outer diameter than both the first needle portion and the second needle portion.

Various example embodiments of the present general inventive concept may provide an injection needle including a hub portion configured to mate with a corresponding hub portion of a medication delivery device, a needle attached at a proximal end to the hub and configured to have a first outer diameter and to extend in a longitudinal direction from the hub, and so as to bend away at a bend point proximate a distal end of the needle such that a portion of the needle at the distal end forms a predetermined angle with the longitudinal direction of the needle on the other side of the point proximate the distal end, and such that the distal end of the needle has a second outer diameter smaller than the first outer diameter to provide a needle tip for injection into soft tissue, and such that a tapering portion is provided to taper the first outer diameter to the second outer diameter, and a ridged portion provided proximate a point at which the tapering portion begins from the first outer diameter, the ridged portion having a third outer diameter larger than the first outer diameter, to form a safety stop for injection of the needle tip.

According to various embodiments of the present general inventive concept, an injection needle including a first needle portion having a first outer diameter, and a second needle portion having a second outer diameter smaller than the first outer diameter and a tip configured to penetrate tissue of a patient to deliver medication, is provided.

It is noted that the simplified diagrams and drawings do not illustrate all the various connections and assemblies of the various components, however, those skilled in the art will understand how to implement such connections and assemblies, based on the illustrated components, figures, and descriptions provided herein, using sound engineering judgment.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

While the present general inventive concept has been illustrated by description of several example embodiments, it is not the intention of the applicant to restrict or in any way limit the scope of the inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings.

1. An injection needle, comprising:
a guide portion configured to have a first outer diameter; and
a delivery portion, configured with a tip to penetrate tissue and deliver a medication, and to have a second outer diameter smaller than the first outer diameter, extending from a distal end of the guide portion;
wherein the guide portion is configured to have a bend proximate the distal end such that the delivery portion extends at a predetermined angle from a line of the guide portion opposite the bend.

2. The injection needle of claim 1, wherein the guide portion is configured to have the first outer diameter at both sides extending from the bend.

3. The injection needle of claim 1, further comprising a hub provided at a proximal end of the guide portion, the hub being configured to mate with a corresponding hub to connect the injection needle to a medication delivery device.

4. The injection needle of claim 1, wherein the guide portion and the delivery portion are formed from a common body.

5. The injection needle of claim 4, wherein the delivery portion is configured to be tapered from the distal end of the guide portion to a predetermined point on the delivery portion.

6. The injection needle of claim 5, further comprising a ridged portion having a third outer diameter larger than the first outer diameter, and provided at the distal end of the guide portion, to serve as a safety stop for the delivery portion.

7. The injection needle of claim 6, wherein the ridged portion is a separate body adhered to the injection needle.

8. The injection needle of claim 7, wherein the ridged portion is formed of a separate material than the injection needle.

9. The injection needle of claim 4, further comprising a ridged portion proximate the distal end of the guide portion, the ridged portion having a third outer diameter larger than the first outer diameter, such that the ridged portion serves as a safety stop for the delivery portion.

10. The injection needle of claim 4, wherein the guide portion is more rigid than the delivery portion.

11. The injection needle of claim 1, wherein the guide portion and the delivery portion are separately formed.

12. The injection needle of claim 11, wherein the delivery portion extends through the entirety of the guide portion, the guide portion being configured as a sleeve surrounding the delivery portion.

13. The injection needle of claim 11, further comprising a ridged portion having a third outer diameter larger than the first outer diameter, and provided proximate the distal end of the guide portion, to serve as a safety stop for the delivery portion.

14. The injection needle of claim 11, wherein the guide portion is more rigid than the delivery portion.

15. The injection needle of claim 11, wherein both the guide portion and the delivery portion are fixed to a hub at a proximal end of the guide portion.

16. The injection needle of claim 11, wherein the delivery portion is adhered to the guide portion.

17. An injection needle, comprising:
a first needle portion having a first outer diameter; and
a second needle portion having a second outer diameter, the second outer diameter being smaller than the first outer diameter,
wherein the second needle portion has a tip configured to penetrate a soft tissue.

18. The injection needle of claim 17, further comprising a hub mounted to the first needle portion, the hub being configured to mate with other similar hubs.

19. The injection needle of claim 17, wherein the second needle portion forms an angle with the first needle portion.

20. The injection needle of claim 19, wherein the angle is approximately 37 degrees.

21. The injection needle of claim 19, wherein the angle is approximately 90 degrees.

22. The injection needle of claim 17, further comprising a safety stop positioned at an opposite end of the second needle portion from the tip, the safety stop having a larger outer diameter than both the first needle portion and the second needle portion.

23. An injection needle, comprising:
a hub portion configured to mate with a corresponding hub portion of a medication delivery device;
a needle attached at a proximal end to the hub and configured to have a first outer diameter extending in a longitudinal direction from the hub, the first outer diameter having a bend portion proximate a distal end of the needle, the bend portion having a predetermined angle matching a corresponding tissue path through which the needle will pass to enter a targeted region of the patient, the needle including a distal end configured to have a second outer diameter smaller than the first outer diameter, the second diameter terminating to a needle tip for delivery of medication into the targeted region, the first diameter being specifically sized to push away bodily tissue in the tissue path a distance sufficient to provide an opening for the second diameter to enter the targeted region at the predetermined angle to deliver medication to the targeted region.

24. The injection needle of claim 23, further including a ridged portion formed proximate a point at which the tapering portion begins from the first outer diameter, the ridged portion having a third outer diameter larger than the first outer diameter specifically sized to abut bodily tissue at an end of the tissue path to inhibit the needle tip from penetrating beyond the targeted region.

25. The injection needle of claim 24, with a suction port to remove and gather excess spilled medication, blood, or secretions, or any combination of these fluids. An injection needle, comprising: a suction port, a conduit tube along the body of the needle, and an exit port. Exit port connects to suction, and thus evacuates all material away from operative site and patient and to a suction, collection device. A tube will connect to exit port. This tube may be either fixed to needle unit or detachable.

26. The injection needle of claim 25, with a conduit along body of needle. Conduit may be attached in any manner to body of needle.

27. The injection needle of claim 26, containing exit port, attaching to tubing, hose, or otherwise to connect with suction port.

28. The injection needle of claim 27, containing hose, tube, or other, either permanently attached to device or detachable. This hose connects needle to external collection or suction device.
